(19) United States
(12) Patent Application Publication
(10) Pub. No.: US 2026/0349281 A1
(43) Pub. Date: Nov. 5, 2026

(54) CORRECTIVE LENS AND METHOD FOR CONTROLLING MYOPIA EFFECTS OR EYE ABERRATIONS

(71) Applicant: UNIVERSITY OF HOUSTON SYSTEM, Houston, TX (US)

(72) Inventor: Geunyoung YOON, Houston, TX (US)

(73) Assignee: UNIVERSITY OF HOUSTON SYSTEM, Houston, TX (US)

(21) Appl. No.: 19/486,088

(22) PCT Filed: May 29, 2024
(86) PCT No.: PCT/US2024/031410

§ 371 Date: Nov. 20, 2025

Related U.S. Application Data
(60) Provisional application No. 63/470,082, filed on May 31, 2023.
(60) Provisional application No. 63/534,749, filed on Aug. 25, 2023.

(51) Int. Cl. G02C 7/16

(52) U.S. Cl. CPC G02C 7/16

(57) ABSTRACT

A corrective lens for controlling myopia of an eye of a patient. The corrective lens includes a central region at least partially preventing light transmission, and a light transmissive region formed at least through a center area of the central region.

### CROSS-REFERENCE TO RELATED APPLICATIONS

This application claims priority to and the benefit of U.S. Provisional Patent Application Ser. No. 63/470,082, filed May 31, 2023, and claims priority to and the benefit of U.S. Provisional Patent Application Ser. No. 63/534,749, filed Aug. 25, 2023, each of which is incorporated herein by reference in its entirety.

### FIELD OF THE INVENTION

The present invention relates generally to a corrective lens for correction of eye disorders, and more specifically, to a corrective lens with an aperture for controlling myopia (e.g., slowing or stopping myopia) or other eye aberrations.

### BACKGROUND OF THE INVENTION

Myopia is one of the leading causes of visual impairment worldwide and is linked to severe eye diseases that can cause permanent blindness. The overall prevalence of myopia has increased substantially in recent years and is estimated to be approaching 50% of the world's population. Although myopia can be corrected with optical and surgical interventions, pathological myopia is known to increase the risk of eye diseases, such as cataracts, glaucoma, and macular degeneration which cause large social economic burden worldwide.

Despite the identification of risk factors for myopia progression, such as age of onset, genetics, visual environment, and peripheral focus, the causes of myopia are not fully understood. For example, optical interventions, such as bifocal/multifocal and orthokeratology (OK) lenses, have been developed based on the idea that inducing peripheral myopic defocus had a positive effect on slowing progression of myopia. However, human-based longitudinal studies have shown almost no correlation between peripheral defocus and myopia development. Thus, the genesis of myopia remains uncertain, but is generally considered to have a multifactorial origin composed of optical, genetic, and environmental factors.

In addition to myopia, it has long been known that optical defects in the eye, such as aberrations, degrade visual performance especially under dim light condition where the eye pupil increases. The degradation becomes even more dominant in highly aberrated eyes with irregular cornea such as keratoconus and post-refractive surgery ectasia. These aberrations cannot be corrected with conventional spectacles or contact lenses especially in moderate to severe pathologic cases. Moreover, some proposed solutions in the form of wavefront-guided or customized specialty contact lenses are severely limited based on commercialization, cost, and other problems. Yet another problem associated with wavefront-guided contact lenses is that correction performance significantly depends on positional stability of the lens.

The present disclosure provides a solution for these and other problems.

### SUMMARY OF THE INVENTION

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

It has recently been shown that there is a correlation between peripheral anisotropic optical blur and myopia development. As such, according to one general aspect of the present disclosure, this correlation is utilized to provide a correction for myopia.

According to certain aspects of the present disclosure, a corrective lens for correction of myopia of an eye of a patient is disclosed. The corrective lens includes a central region at least partially preventing light transmission, and a light transmissive region formed at least through a center area of central region.

According to certain aspects of the present disclosure, the central region is circular.

According to certain aspects of the present disclosure, the light transmissive region is circular.

According to certain aspects of the present disclosure, the central region has a radial gradient of opacity allowing more transmission of light in proximity to the light transmissive region.

According to certain aspects of the present disclosure, the central region has a circumferentially asymmetric radial gradient of opacity allowing more transmission of light in proximity to the light transmissive region.

According to certain aspects of the present disclosure, the light transmissive region is radially symmetrical.

According to certain aspects of the present disclosure, the central region has a radial gradient of opacity allowing more transmission of light in proximity to the light transmissive region.

According to certain aspects of the present disclosure, the light transmissive region has an elongate shape.

According to certain aspects of the present disclosure, the corrective lens has an orientation when worn on an eye, and the light transmissive region is elongate in a horizontal direction relative to the orientation of the corrective lens when worn.

According to certain aspects of the present disclosure, the corrective lens has an orientation when worn on an eye, and the light transmissive region is elongate in a vertical direction relative to the orientation of the corrective lens when worn.

According to certain aspects of the present disclosure, the corrective lens has an orientation when worn on an eye, and the light transmissive region is elongate in a diagonal direction relative to the orientation of the corrective lens when worn.

According to certain aspects of the present disclosure, the corrective lens is manufactured by printing the central region onto a surface of the corrective lens.

According to certain aspects of the present disclosure, the light transmissive region comprises an aperture through the corrective lens.

According to certain aspects of the present disclosure, the corrective lens is manufactured by including an opaque pigment in material of the corrective lens, and subsequently forming the aperture through the corrective lens.

According to certain aspects of the present disclosure, the aperture is formed through the corrective lens with a laser.

According to certain aspects of the present disclosure, the corrective lens is selected from the group consisting of: a soft contact lens, a hard contact lens, a spectacle lens, and an intraocular lens.

According to certain aspects of the present disclosure, a method of correction of myopia comprises positioning a corrective lens to an eye. The corrective lens has a central region at least partially preventing light transmission, and a light transmissive region formed at least through the center of central region.

According to other aspects of the present disclosure, a corrective lens is directed to corrections of aberrations of an eye of a patient. The lens includes a central region formed in the center of the lens. The lens prevents light transmission. The lens further includes a slit aperture formed in the center of the central region. The slit allows light transmission through the slit aperture.

According to some aspects of the present disclosure, the corrective lens disclosed above improves depth of focus, which improves retinal image quality of objects that are at far, intermediate, and/or near distances from the eye.

According to some aspects of the present disclosure, the corrective lens disclosed above is one of a soft contact lens, a hard contact lens, spectacles, and/or an intraocular lens.

According to some aspects of the present disclosure, the slit aperture disclosed above is a horizontal slit.

According to some aspects of the present disclosure, the slit aperture disclosed above is a vertical slit.

According to some aspects of the present disclosure, the slit aperture disclosed above is angled relative to a horizontal plane.

According to some aspects of the present disclosure, the slit aperture disclosed above has an oval shape.

According to some aspects of the present disclosure, the slit aperture disclosed above is decentered slightly from the geometric center of the lens, depending on the lens position on the eye relative to a pupil center of the eye.

According to some aspects of the present disclosure, the corrective lens disclosed above is configured to correct spherical and cylindrical refractive error induced by the corrective lens.

According to yet other aspects of the present disclosure, a method is directed to controlling aberrations of an eye. The method includes positioning a corrective lens to an eye. The corrective lens has a central region formed in the center of the lens to prevent light transmission. The corrective lens further has a slit aperture formed in the center of a central region. The corrective lens allows light transmission through the slit aperture.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The disclosure, and its advantages and drawings, will be better understood from the following description of representative embodiments together with reference to the accompanying drawings. These drawings depict only representative embodiments and are therefore not to be considered as limitations on the scope of the various embodiments or claims.

FIG. 1A is a peripheral retinal image of a point spread function subjected to an applied defocus of minus 2 diopters (D), according to an aspect of the present disclosure.

FIG. 1B is a peripheral retinal image of a point spread function subjected to an applied defocus of minus 1.75 D, according to an aspect of the present disclosure.

FIG. 1C is a peripheral retinal image of a point spread function subjected to an applied defocus of minus 1.5 D, according to an aspect of the present disclosure.

FIG. 1D is a peripheral retinal image of a point spread function subjected to an applied defocus of minus 1.25 D, according to an aspect of the present disclosure.

FIG. 1E is a peripheral retinal image of a point spread function subjected to an applied defocus of minus 1 D, according to an aspect of the present disclosure.

FIG. 1F is a peripheral retinal image of a point spread function subjected to an applied defocus of minus 0.75 D, according to an aspect of the present disclosure.

FIG. 1G is a peripheral retinal image of a point spread function subjected to an applied defocus of minus 0.5 D, according to an aspect of the present disclosure.

FIG. 1H is a peripheral retinal image of a point spread function subjected to an applied defocus of minus 0.25 D, according to an aspect of the present disclosure.

FIG. 1I is a peripheral retinal image of a point spread function subjected to an applied defocus of 0 D, according to an aspect of the present disclosure.

FIG. 1J is a peripheral retinal image of a point spread function subjected to an applied defocus of 0.25 D, according to an aspect of the present disclosure.

FIG. 1K is a peripheral retinal image of a point spread function subjected to an applied defocus of 0.5 D, according to an aspect of the present disclosure.

FIG. 2A is a peripheral retinal image of a point spread function subjected to an applied defocus of minus 2 D and further including a central aperture of about 1.5 millimeters (mm), according to an aspect of the present disclosure.

FIG. 2B is a peripheral retinal image of a point spread function subjected to an applied defocus of minus 1.75 D and further including a central aperture of about 1.5 mm, according to an aspect of the present disclosure.

FIG. 2C is a peripheral retinal image of a point spread function subjected to an applied defocus of minus 1.5 D and further including a central aperture of about 1.5 mm, according to an aspect of the present disclosure.

FIG. 2D is a peripheral retinal image of a point spread function subjected to an applied defocus of −1.25 D and further including a central aperture of about 1.5 mm, according to an aspect of the present disclosure.

FIG. 2E is a peripheral retinal image of a point spread function subjected to an applied defocus of minus 1 D and further including a central aperture of about 1.5 mm, according to an aspect of the present disclosure.

FIG. 2F is a peripheral retinal image of a point spread function subjected to an applied defocus of minus 0.75 D and further including a central aperture of about 1.5 mm, according to an aspect of the present disclosure.

FIG. 2G is a peripheral retinal image of a point spread function subjected to an applied defocus of minus 0.5 D and further including a central aperture of about 1.5 mm, according to an aspect of the present disclosure.

FIG. 2H is a peripheral retinal image of a point spread function subjected to an applied defocus of minus 0.25 D and further including a central aperture of about 1.5 mm, according to an aspect of the present disclosure.

FIG. 2I is a peripheral retinal image of a point spread function subjected to an applied defocus of 0 D and further including a central aperture of about 1.5 mm, according to an aspect of the present disclosure.

FIG. 2J is a peripheral retinal image of a point spread function subjected to an applied defocus of 0.25 D and further including a central aperture of about 1.5 mm, according to an aspect of the present disclosure.

FIG. 2K is a peripheral retinal image of a point spread function subjected to an applied defocus of 0.5 D and further including a central aperture of about 1.5 mm, according to an aspect of the present disclosure.

FIG. 3A is a peripheral retinal image of a point spread function subjected to an applied defocus of minus 2 D and further including an elongate slot aperture of about 1.5×4 mm, according to an aspect of the present disclosure.

FIG. 3B is a peripheral retinal image of a point spread function subjected to an applied defocus of minus 1.75 D and further including an elongate slot aperture of about 1.5×4 mm, according to an aspect of the present disclosure.

FIG. 3C is a peripheral retinal image of a point spread function subjected to an applied defocus of minus 1.5 D and further including an elongate slot aperture of about 1.5×4 mm, according to an aspect of the present disclosure.

FIG. 3D is a peripheral retinal image of a point spread function subjected to an applied defocus of minus 1.25 D and further including an elongate slot aperture of about 1.5×4 mm, according to an aspect of the present disclosure.

FIG. 3E is a peripheral retinal image of a point spread function subjected to an applied defocus of minus 1 D and further including an elongate slot aperture of about 1.5×4 mm, according to an aspect of the present disclosure.

FIG. 3F is a peripheral retinal image of a point spread function subjected to an applied defocus of minus 0.75 D and further including an elongate slot aperture of about 1.5×4 mm, according to an aspect of the present disclosure.

FIG. 3G is a peripheral retinal image of a point spread function subjected to an applied defocus of minus 0.5 D and further including an elongate slot aperture of about 1.5×4 mm, according to an aspect of the present disclosure.

FIG. 3H is a peripheral retinal image of a point spread function subjected to an applied defocus of minus 0.25 D and further including an elongate slot aperture of about 1.5×4 mm, according to an aspect of the present disclosure.

FIG. 3I is a peripheral retinal image of a point spread function subjected to an applied defocus of 0 D and further including an elongate slot aperture of about 1.5×4 mm, according to an aspect of the present disclosure.

FIG. 3J is a peripheral retinal image of a point spread function subjected to an applied defocus of 0.25 D and further including an elongate slot aperture of about 1.5×4 mm, according to an aspect of the present disclosure.

FIG. 3K is a peripheral retinal image of a point spread function subjected to an applied defocus of 0.5 D and further including an elongate slot aperture of about 1.5×4 mm, according to an aspect of the present disclosure.

FIG. 4 is a plan view of an exemplary corrective lens, according to certain aspects of the present disclosure.

FIG. 5 is a plan view of another exemplary corrective lens, according to certain aspects of the present disclosure.

FIG. 6 is a plan view of a further exemplary corrective lens, according to certain aspects of the present disclosure.

FIG. 7 is a plan view of yet another exemplary corrective lens, according to certain aspects of the present disclosure.

FIG. 8A illustrates simulated retinal image quality for an eye having aberration and coma provided by a lens having a circular aperture without and with defocus and astigmatism optimization, according to certain aspects of the present disclosure.

FIG. 8B illustrates simulated retinal image quality for an eye having aberration and coma provided by a lens having a horizontal slit aperture without and with defocus and astigmatism optimization, according to certain aspects of the present disclosure.

FIG. 8C illustrates simulated retinal image quality for an eye having aberration and coma provided by a lens having a vertical slit aperture without and with defocus and astigmatism optimization, according to certain aspects of the present disclosure.

FIG. 9A illustrates simulated retinal image quality for an eye having aberration and coma subjected to an applied defocus of minus 1 D for lens having a circular aperture, a horizontal slot aperture, and a vertical slot aperture, according to an aspect of the present disclosure.

FIG. 9B illustrates simulated retinal image quality for an eye having aberration and coma subjected to an applied defocus of minus 0.75 D for lens having a circular aperture, a horizontal slot aperture, and a vertical slot aperture, according to an aspect of the present disclosure.

FIG. 9C illustrates simulated retinal image quality for an eye having aberration and coma subjected to an applied defocus of minus 0.5 D for lens having a circular aperture, a horizontal slot aperture, and a vertical slot aperture, according to an aspect of the present disclosure.

FIG. 9D illustrates simulated retinal image quality for an eye having aberration and coma subjected to an applied defocus of minus 0.25 D for lens having a circular aperture, a horizontal slot aperture, and a vertical slot aperture, according to an aspect of the present disclosure.

FIG. 9E illustrates simulated retinal image quality for an eye having aberration and coma subjected to an applied defocus of 0 D for lens having a circular aperture, a horizontal slot aperture, and a vertical slot aperture, according to an aspect of the present disclosure.

FIG. 9F illustrates simulated retinal image quality for an eye having aberration and coma subjected to an applied defocus of 0.25 D for lens having a circular aperture, a horizontal slot aperture, and a vertical slot aperture, according to an aspect of the present disclosure.

FIG. 9G illustrates simulated retinal image quality for an eye having aberration and coma subjected to an applied defocus of 0.5 D for lens having a circular aperture, a horizontal slot aperture, and a vertical slot aperture, according to an aspect of the present disclosure.

FIG. 9H illustrates simulated retinal image quality for an eye having aberration and coma subjected to an applied defocus of 0.75 D for lens having a circular aperture, a horizontal slot aperture, and a vertical slot aperture, according to an aspect of the present disclosure.

FIG. 9I illustrates simulated retinal image quality for an eye having aberration and coma subjected to an applied defocus of 1 D for lens having a circular aperture, a horizontal slot aperture, and a vertical slot aperture, according to an aspect of the present disclosure.

### DETAILED DESCRIPTION

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively. Additionally, words of direction, such as “top,” “bottom,” “left,” “right,” “above,” and “below” are intended to relate to the equivalent direction as depicted in a reference illustration; as understood contextually from the object(s) or element(s) being referenced, such as from a commonly used position for the object(s) or element(s); or as otherwise described herein.

Uncorrected, or corrected (e.g., by monofocal or bifocal lenses), human vision typically provides an in-focus image near the center of vision of the eye and a blurred image towards the periphery of the eye. The center of vision is typically within about 10°of our line of sight, which is defined to be at about 0°. Along the line of sight, an image is projected directly onto the fovea of the eye. Peripheral vision is typically from an angle away from the line of sight (commonly called eccentricity) of more than about 10° out to about 90°.

Peripheral blur anisotropy is induced by asymmetric wavefront aberrations such as astigmatism and coma. Intrinsic peripheral aberrations of a human eye induce directionally oriented (anisotropic) blur in images formed on the peripheral retina. The primary goal of any corrective measure is to correct the vision of a person to compensate for optical defects of the eye, known as aberrations. In particular, the primary goal of any measure of myopia correction is to correct the vision to compensate for the myopia by providing an in-focus image in the forward line of sight. Corrective measures include, for example without limitation, contact lenses, including soft contact lenses (SCL), hard contact lenses, eyeglasses, and surgical interventions, including writing lenses directly on the cornea of the eye. Hard contact lenses include, for example, rigid gas permeable, scleral lens (small and large diameter), and orthokeratology. However, conventional myopia correction methods, while effective at controlling myopia, do not prevent the intrinsic peripheral aberrations of the eye from inducing anisotropic blur in images formed on the peripheral retina. Controlling myopia includes, for example, correcting, slowing down, or stopping myopia.

The lack of prevention of intrinsic peripheral aberrations is an important consideration because the anisotropic blur (also called optical anisotropy) drives excessive eye growth, effectively causing myopia. Particularly for children and young adults where eye growth is still active, it is therefore desirable when controlling myopia, to also modify the peripheral vision by intentionally introducing lower and higher order aberrations to corrective measures. These higher order aberrations can be introduced by a technique called wavefront-guided technology, which reduces anisotropic blur on a peripheral retina of an eye while not affecting images in the forward line of sight. Controlling the aberrations in the peripheral vision may not per se improve peripheral vision, but rather it can control growth of the eye, particularly in young people, which can minimize or possibly prevent the disorders commonly associated with myopia.

The present disclosure is at least in part directed toward an exemplary method that improves retinal image quality to reduce the impact of aberrations of the eye to improve visual performance. The eye's aberration increases with the eye's pupil size, i.e., the smaller the pupil diameter, the better the retinal image quality. Therefore, using a very small circular aperture is an effective way to reduce the effects of the aberrations. However, considerable loss in the amount of light entering the eye is a serious concern especially under dark visual environment conditions. An embodiment herein addresses this problem. For example, an embodiment herein uses an elongate shape, for example, an elongate slot that increases the amount of the light entering the eye while maintaining the small aperture effect, i.e., reducing the aberration effects.

The small circular aperture and the slit or elongate shaped aperture could be implemented into a contact lens (soft and hard), spectacles and potentially an intraocular lens. The small circular aperture and the slit or elongate shaped aperture are effective in extending depth of focus along the line of sight (along the fovea), resulting in improving near vision in these patients suffering from presbyopia (age-related near vision loss). Thus, the small circular aperture and the slit or elongate shaped aperture improve retinal image quality of objects that are at far, intermediate and near distances from the eye.

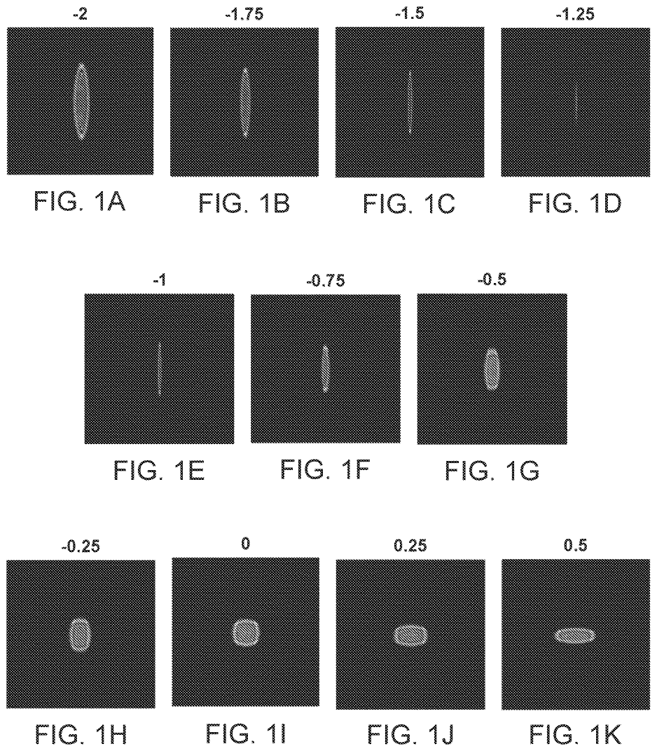

A strategy to slow down and potentially stop myopia progression is to reduce blur anisotropy across the peripheral visual field. Referring to FIGS. 1A-1K, a series of images of a point spread function on a peripheral retina is presented. Each point spread function is subject to an applied defocus as indicated by the number above each image given in diopters (D). Starting at FIG. 1A, the defocus is minus 2.0 D and increases in increments of 0.25 D ending at FIG. 1K, which is a point spread function on the peripheral retina presented for a defocus of 0.5 D.

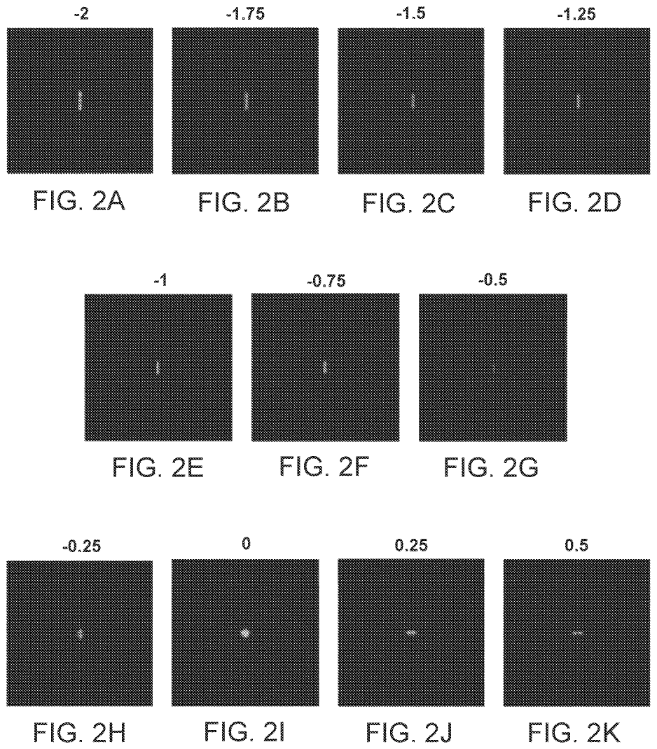

FIGS. 2A-2K show the point spread functions on a peripheral retina for the same levels of defocus as in FIGS. 1A-1K but further including a central aperture of about 1.5 mm in diameter. The blurred images on the peripheral retina of the images in FIGS. 2A-2K are smaller than the corresponding blurred images in FIGS. 1A-1K.

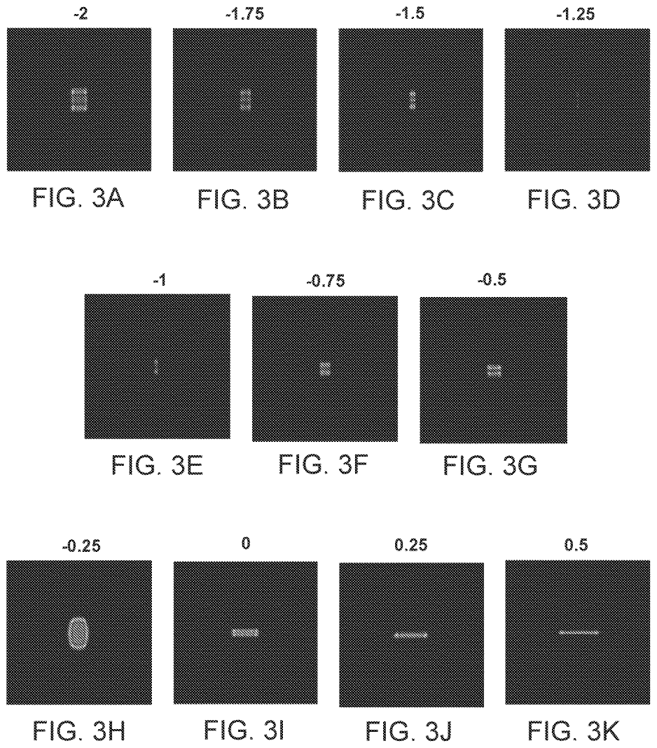

FIGS. 3A-3K show images on a peripheral retina for the same levels of defocus as in FIGS. 1A-1K but further including an elongate slot aperture of about 1.5×4 mm in size. The blurred images on the peripheral retina in FIGS. 3A-3K are smaller than the corresponding blurred images in FIGS. 1A-1K, but are slightly larger than the corresponding blurred images in FIGS. 2A-2K.

The central aperture and the elongate slot aperture correct previously uncorrected natural peripheral asymmetric aberrations. As is demonstrated in FIGS. 1-3, the central aperture and the elongate slot aperture affect light passing through them to make the point spread function (PSF) at the peripheral retina smaller for less blur anisotropy at the periphery of vision (i.e., at higher eccentricity). Moreover, the corrections to the peripheral aberrations optimally can be effective not just at the end of the peripheral vision towards a 30° eccentricity, but also at 20° eccentricity, and ideally across a wide visual field. Generally, the approach is to provide additional corrections beyond myopia to provide a reduced radially asymmetric blur in the peripheral vision. As is demonstrated herein, the desired higher order peripheral aberrations can be included in the corrective optical design of any suitable myopia correction device by the addition of the small circular aperture or the elongate slot aperture.

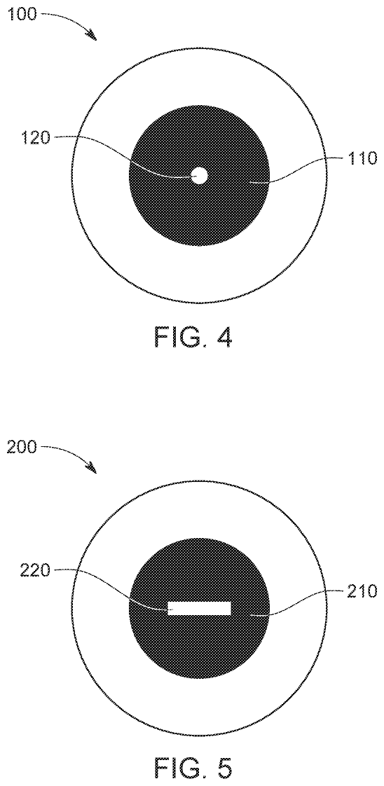

Referring to FIG. 4, in an embodiment a corrective lens 100 comprises an opaque central region 110 at least partially preventing light transmission and a light transmissive region 120 formed at least through the center of central region 110. In an embodiment a light transmissive region 120 is a transparent portion 120 of the corrective lens 100. In another embodiment the light transmissive region 120 is an aperture 120 disposed through the corrective lens 100. Such an aperture 120, for example, as a small hole, could be implemented into a corrective lens 100, for example, a contact lens (soft or hard), spectacles, or an intraocular lens. In an embodiment the central region 110 is circular. In an embodiment the light transmissive region 120 is circular.

Peripheral blur anisotropy becomes larger with increasing the eye's pupil size. Therefore, to reduce the peripheral blur anisotropy and thereby slow down myopia progression, the light transmissive region 120 is as small as possible to allow enough light to pass through to the retina to ensure that an image formed on the retina is not too dim. A comparison of FIGS. 2A-2K to FIGS. 1A-1K shows the effect of a small light transmissive region 120 on the blurred images on a peripheral retina.

Referring to FIG. 5, in an embodiment a corrective lens 200 comprises an opaque central region 210 at least partially preventing light transmission and a light transmissive region 220 formed at least through the center of central region 210. In this embodiment, to ensure that enough light passes through to the retina, the light transmissive region 220 is larger than the aperture 120 and has a radially symmetrical elongate shape 220, shown in particular as an elongate slot 220. The corrective lens 200 can be made to have a predetermined orientation when worn on an eye. For example, by making the corrective lens 200 heavier along an edge portion, after being put on and being allowed to settle, the corrective lens 200 rotationally settles into the predetermined orientation relative to the eye. In an embodiment the corrective lens 200 has an orientation when worn on an eye, and the light transmissive region 220 is elongate in a horizontal direction relative to the orientation of the corrective lens 220 when worn. In other embodiments, the corrective lens 200 has an orientation when worn on an eye, and the light transmissive region 220 is elongate in a vertical or diagonal direction relative to the orientation of the corrective lens 220 when worn.

The elongate slot 220 effectively increases the amount of light transmitted to the peripheral retina when compared with the aperture 120. However, reduction of the peripheral blur anisotropy is less effective as can be seen by comparison of FIGS. 3A-3K to FIGS. 2A-2K. This comparison shows the blurred images on the peripheral retina in FIGS. 3A to 3K are somewhat larger than the corresponding blurred images on the peripheral retina shown in FIGS. 2A-2K.

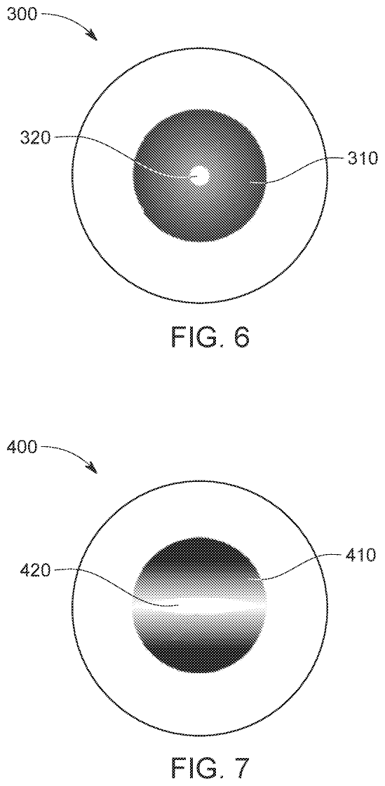

Referring to FIG. 6, in an embodiment, instead of having an opaque central region 110, as illustrated for the embodiment in FIG. 1, light transmission through the corrective lens 300 could gradually vary from 100% at the light transmissive region 320 to a lower percentage of transmission or to being entirely opaque (0% transmission) at some distance from the center of the central region 310. Stated another way, in the embodiment of the corrective lens 300, the central region 310 has a radial gradient of opacity allowing more transmission of light in proximity to the light transmissive region 320.

Referring to FIG. 7, in an embodiment, instead of having an opaque central region 210, as illustrated for the embodiment in FIG. 2, light transmission through the corrective lens 400 could gradually vary from 100% at the light transmissive region 420 to a lower percentage of transmission or to being opaque (0% transmission) at some distance from the center of the central region 410. Stated another way, in the embodiment of the corrective lens 400, the central region 410 has a radial gradient of opacity allowing more transmission of light in proximity to the light transmissive region 420.

In an embodiment, a method for treatment of myopia comprises positioning a corrective lens 100, 200, 300, 400 to an eye. The corrective lens 100, 200, 300, 400 comprises at least a central region 110, 210, 310, 410 at least partially preventing light transmission, and a light transmissive region 120, 220, 320, 420 formed at least through the center of central region 110, 210, 310, 410.

For either of the embodiments of the corrective lens 300, 400, the radial gradient of opacity in the central region 310, 410, respectively, can be circumferentially symmetrical, radially symmetrical, or radially asymmetrical. In this context, a circle is a circumferentially symmetrical shape, having the same radius at any point on the circle. Similarly, a circumferentially symmetric radial gradient varies at the same rate in a direction away from the center regardless of circumferential position. Radial symmetry requires only that a portion of a given pattern can be folded over upon itself radially. For example, any even sided polygon is radially symmetrical. In this context, the elongate slot 220, 420 is radially symmetrical. Further in this context, an asymmetric radial gradient does not exhibit radial symmetry.

In an embodiment, any of the corrective lenses 100, 200, 300, 400 described hereinabove can be manufactured by printing the central region 110, 210, 310, 410 onto a surface of the corrective lens 100, 200, 300, 400, respectively. In an embodiment, the corrective lenses 100, 200 are manufactured by including an opaque pigment in material of the central region 110, 210, 310, 410 while not including the opaque pigment in material of the light transmissive region 120, 220, 320, 420. In an embodiment, the corrective lenses 100, 200 are manufactured by including an opaque pigment in material of at least the central region 110, 210 of the corrective lens 100, 200, and subsequently forming the aperture 120, 220 through the corrective lens 100, 200. In an embodiment, the aperture 120, 220 is formed through the corrective lens 100, 200, respectively. The structure described herein for the corrective lens 100, 200, 300, 400 is applicable to any of a soft contact lens, a hard contact lens, a spectacle lens, and an intraocular lens.

According to various embodiments, suitable devices to introduce the peripheral aberrations described hereinabove or to remedy aberrations described hereinabove can be manufactured by any suitable materials by mechanical machining means, such as by use of lathes, milling machines (typically computer numerical control milling (CNC)), etc. Other suitable manufacturing techniques include laser manufacturing techniques. Exemplary suitable laser manufacturing and forming techniques include the blue-IRIS, or blue intra-tissue refractive index shaping. Suitable materials include any suitable plastic, glass, including any suitable contact lens materials.

Software for modeling and creating the structures (e.g., lens patterns to be written by laser or milled by machines) for writing or machining the peripheral aberrations described hereinabove to optical devices (e.g., eyeglass lenses, contact lenses, the cornea of the human eye) can be provided on a computer readable non-transitory storage medium. A computer readable nontransitory storage medium as non-transitory data storage includes any data stored on any suitable media in a non-fleeting manner. Such data storage includes any suitable computer readable nontransitory storage medium, including, but not limited to hard drives, non-volatile RAM, SSD devices, CDs, DVDs, etc.

The apertures and slots 120, 220, 320, 420 in the corrective lens 100, 200, 300, 400 disclosed herein may be provided by either printing a pattern with the aperture 120, 220, 320, 420 on the corrective lens 100, 200, 300, 400 surface, using an inkjet printing process for the pattern with the aperture 120, 220, 320, 420, or depositing a color pigment into the clear corrective lens 100, 200, 300, 400 material and forming the aperture 120, 220, 320, 420 in the color pigment. In the example of an inkjet printer, the pattern may be printed on a corrective lens 100, 200, 300, 400 positioned on a carrier based on a graphic image stored on a computer that controls the inkjet printer. Applications executed on the computer may be used to provide the shape of the aperture 120, 220, 320, 420 as well as the size and opacity of the surrounding central region 110, 210, 310, 410. In an embodiment the position of the aperture 120, 220, 320, 420 could be decentered slightly from the geometric center of the corrective lens 100, 200, 300, 400 depending on the position of the corrective lens 100, 200, 300, 400 on an eye relative to the pupil center of the eye. In addition, as noted above, in various embodiments, the elongate slot 220, 420 is vertically, horizontally, or diagonally oriented relative to the corrective lens 100, 200, 300, 400 when seated on the eye. In an embodiment, the shape of the elongate slot 220, 420 is rectangular, but in other embodiments the shape of the elongate slot 220, 420 comprises or other shapes, for example without limitation, an oval.

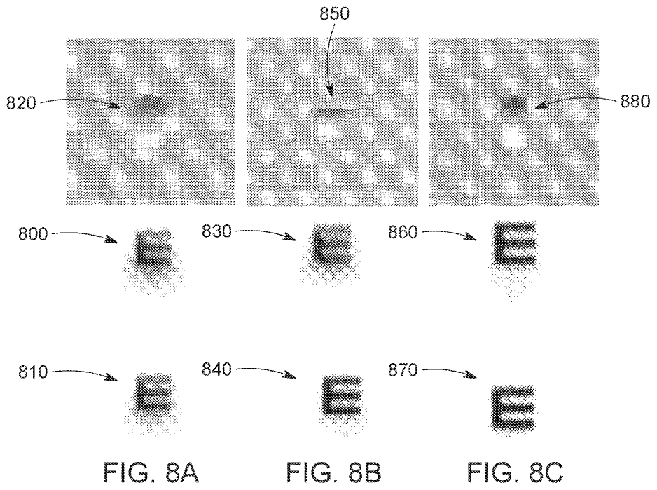

In accordance with a theoretical simulation, an aberration, vertical coma, which is the most common higher-order aberration, measured in keratoconus eyes was used to represent a highly aberrated eye. Referring to FIGS. 8A-8C, retinal image quality was then calculated for 3 different conditions; a typical lens without an aperture (control), a lens with horizontally oriented slit aperture and a lens with vertically oriented slit aperture. Improvements in retinal image quality with both apertures were achieved compared to the control lens. The difference in retinal image quality becomes even more substantial after further correcting spherical and cylindrical refractive error induced by each lens.

Referring to FIGS. 8A-8C, it has been demonstrated that slit apertures reduce the detrimental impact of an eye's aberrations on retinal image quality. FIGS. 8A-8C present images for an eye having a 6 mm pupil, moderate keratoconus, and vertical coma (Z7)=−2 μm. FIG. 8A illustrates simulated retinal image quality for the eye provided by a lens having a 4 mm circular aperture 820 without defocus and astigmatism optimization 800 and with defocus and astigmatism optimization 810. FIG. 8B illustrates simulated retinal image quality for the eye provided by a lens having a horizontal 4×2 mm slit aperture 850 without defocus and astigmatism optimization 830 and with defocus and astigmatism optimization 840. FIG. 8C illustrates simulated retinal image quality for the eye provided by a lens having a 2×4 mm vertical slit aperture 880 without defocus and astigmatism optimization 860 and with defocus and astigmatism optimization 870.

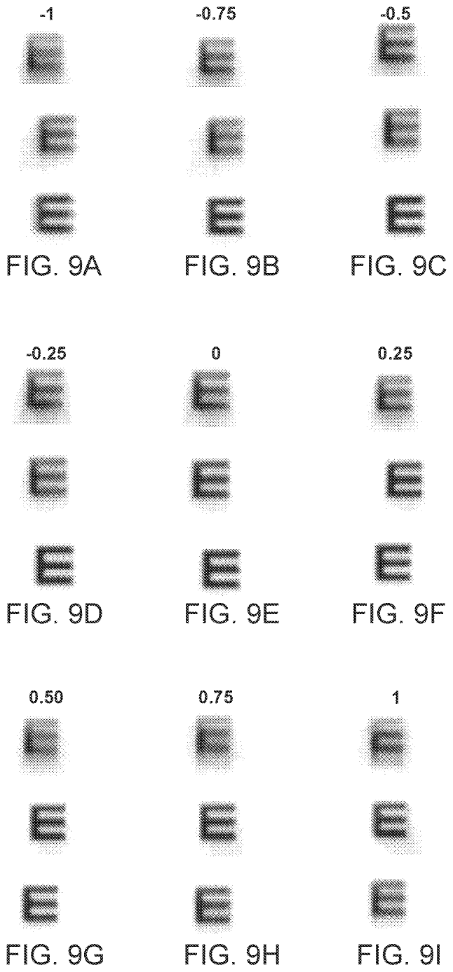

Referring to FIGS. 9A-9I, yet another simulation result has demonstrated that slit apertures also increase depth of focus compared to the conventional lens without a slit, and can be an effective approach to presbyopia correction. FIGS. 9A-9I present images for an eye having a 6 mm pupil, moderate keratoconus, and vertical coma (Z7)=−2 μm.

Referring to FIGS. 9A-9I, a series of sets of three images of retinal image quality is presented. The top image in each set is a simulated retinal image for the eye provided by a lens having a 4 mm circular aperture. The middle image in each set is a simulated retinal image for the eye provided by a lens having a horizontal 4×2 mm slit aperture. The bottom image in each set is a simulated retinal image for the eye provided by a lens having a 2×4 mm vertical slit aperture. Each set of three images is subject to an applied defocus as indicated by the number above each set given in diopters (D). Starting at FIG. 9A, the defocus is minus 1 D and increases in increments of 0.25 D ending at FIG. 9I, for which the defocus of 1 D. In this particular aberration example, as can be seen by comparison of the images in FIGS. 9A-9I, a vertically oriented slit provides even better performance than a horizontally oriented slit in terms of retinal image quality at each object distance as well as depth of focus.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present disclosure have been described above, it should be understood that they have been presented by way of example only, and not limitation. Numerous changes to the disclosed embodiments can be made in accordance with the disclosure herein, without departing from the spirit or scope of the disclosure. Thus, the breadth and scope of the present disclosure should not be limited by any of the above described embodiments. Rather, the scope of the disclosure should be defined in accordance with the following claims and their equivalents.

1. A corrective lens for correcting myopia of an eye of a patient, the corrective lens comprising:
a central region that at least partially prevents light transmission; and
a light transmissive region formed at least through a center area of the central region.

2. The corrective lens of claim 1, wherein the central region is circular.

3. The corrective lens of claim 1, wherein the light transmissive region is circular.

4. The corrective lens of claim 3, wherein the central region has a radial gradient of opacity allowing more transmission of light in proximity to the light transmissive region.

5. The corrective lens of claim 3, wherein the central region has a circumferentially asymmetric radial gradient of opacity allowing more transmission of light in proximity to the light transmissive region.

6. The corrective lens of claim 1, wherein the light transmissive region is radially symmetrical.

7. The corrective lens of claim 6, wherein the central region has a radial gradient of opacity allowing more transmission of light in proximity to the light transmissive region.

8. The corrective lens of claim 6, wherein the light transmissive region has an elongate shape.

9. The corrective lens of claim 8, wherein the corrective lens has an orientation when worn on an eye, and the light transmissive region is elongate in a horizontal direction relative to the orientation of the corrective lens when worn.

10. The corrective lens of claim 8, wherein the corrective lens has an orientation when worn on an eye, and the light transmissive region is elongate in a vertical direction relative to the orientation of the corrective lens when worn.

11. The corrective lens of claim 8, wherein the corrective lens has an orientation when worn on an eye, and the light transmissive region is elongate in a diagonal direction relative to the orientation of the corrective lens when worn.

12. The corrective lens of claim 1, wherein the central region is printed onto a surface of the corrective lens.

13. The corrective lens of claim 1, wherein the light transmissive region includes an aperture through the corrective lens.

14. The corrective lens of claim 13, wherein the corrective lens includes an opaque pigment in a material of the corrective lens, the aperture being formed subsequent to the opaque pigment being added.

15. The corrective lens of claim 13, wherein the aperture is formed through the corrective lens with a laser.

16. The corrective lens of claim 1, wherein the corrective lens is selected from the group consisting of a soft contact lens, a hard contact lens, a spectacle lens, and an intraocular lens.

17. A method of controlling myopia, the method comprising positioning a corrective lens to an eye, the corrective lens having a central region at least partially preventing light transmission, the corrective lens further having a light transmissive region formed at least through a center area of the central region.
